(19) United States
(12) Patent Application Publication
(10) Pub. No.: US 2017/0266368 A1
(43) Pub. Date: Sep. 21, 2017

(54) COUPLING INTERFACE FOR LOCKING AN ACTUATOR OF A FLUID INFUSION DEVICE TO A STOPPER OF A FLUID RESERVOIR

(71) Applicant: MEDTRONIC MINIMED, INC., Northridge, CA (US)

(72) Inventor: Jacob E. Pananen, Santa Monica, CA (US)

(21) Appl. No.: 15/071,013

(22) Filed: Mar. 15, 2016

(51) Int. Cl. A61M 5/145; A61M 5/142

(52) U.S. Cl. CPC A61M 5/1452; A61M 5/142; A61M 5/1456; A61M 2205/00; A61M 2202/04

(57) ABSTRACT

An assembly for an infusion device that delivers fluid to a user is disclosed. The assembly includes a reservoir and a piston actuator. The reservoir includes a body section having a fluid chamber, a fitting section extending from the body section, a housing lock structure to couple the reservoir to a housing of the infusion device, and a piston located within the fluid chamber. The housing lock structure inhibits axial movement of the reservoir relative to the housing. The piston is movable relative to the body section, and it has a tip section facing the fluid chamber and a base section opposite the tip section. The base section has an actuator lock structure. The piston actuator controls axial movement of the piston within the fluid chamber, and it has a piston lock structure that cooperates with the actuator lock structure to removably couple the piston to the piston actuator.

### TECHNICAL FIELD

Embodiments of the subject matter described herein relate generally to fluid infusion devices such as personal insulin infusion pumps. More particularly, embodiments of the subject matter relate to fluid reservoir and connector features of a fluid infusion device.

### BACKGROUND

Portable medical devices are useful for patients that have conditions that must be monitored on a continuous or frequent basis. For example, diabetics are usually required to modify and monitor their daily lifestyle to keep their body in balance, in particular, their blood glucose (BG) levels. Individuals with Type 1 diabetes and some individuals with Type 2 diabetes use insulin to control their BG levels. To do so, diabetics routinely keep strict schedules, including ingesting timely nutritious meals, partaking in exercise, monitoring BG levels daily, and adjusting and administering insulin dosages accordingly.

The prior art includes a number of fluid infusion devices and insulin pump systems that are designed to deliver accurate and measured doses of insulin via infusion sets (an infusion set delivers the insulin through a small diameter tube that terminates at, e.g., a cannula inserted under the patient's skin). In lieu of a traditional syringe, the patient can simply activate the insulin pump to administer an insulin bolus as needed, for example, in response to the patient's current BG level.

A typical infusion pump includes a housing, which encloses a pump drive system, a fluid containment assembly, an electronics system, and a power supply. The pump drive system typically includes a small motor (DC, stepper, solenoid, or other varieties) and drive train components such as gears, screws, and levers that convert rotational motor motion to a translational displacement of a piston in a reservoir, which may be in the form of a user-filled reservoir. The fluid containment assembly typically includes the reservoir with the piston, tubing, and a catheter or infusion set to create a fluid path for carrying medication from the reservoir to the body of a user. The electronics system regulates power from the power supply to the motor. The electronics system may include programmable controls to operate the motor continuously or at periodic intervals to obtain a closely controlled and accurate delivery of the medication over an extended period.

The fluid reservoir of an infusion pump can be filled by way of an interface component (such as a transfer guard) that establishes a fluid connection between the reservoir and a sterile vial of the medication fluid. The piston of the reservoir is manually actuated to pressurize the vial, extract medication fluid from the vial, and remove air bubbles from the reservoir. After the reservoir is filled, a cap of an infusion set component is attached to the neck of the reservoir. The cap also serves to secure the reservoir into the infusion pump housing. An alternative reservoir filling methodology employs a devoted filling device that accepts the empty reservoir and the vial of medication fluid, and automatically fills the reservoir. Use of a filling device eliminates some of the manual steps, but the user still needs to manipulate the reservoir and move it from the filling device to the infusion pump.

Accordingly, it is desirable to have a different methodology for filling a fluid reservoir of a fluid infusion device, such as an insulin pump. In addition, it is desirable to have fluid reservoir, fluid connector, and infusion device features and structures that support different filling methodologies. Furthermore, other desirable features and characteristics will become apparent from the subsequent detailed description and the appended claims, taken in conjunction with the accompanying drawings and the foregoing technical field and background.

### BRIEF SUMMARY

A fluid reservoir for a fluid infusion device is disclosed herein. The fluid infusion device delivers medication fluid to a user, and an exemplary embodiment of the fluid reservoir includes a primary body section having a fluid chamber defined therein, a fitting section extending from the primary body section, a housing lock structure, and a piston located within the fluid chamber. The housing lock structure is configured to removably couple the fluid reservoir to a housing of the fluid infusion device. The housing lock structure inhibits axial movement of the fluid reservoir relative to the housing. The piston is axially movable relative to the primary body section. The piston includes a tip section facing the fluid chamber and a base section opposite the tip section. The base section includes an actuator lock structure to removably couple the piston to a piston actuator of the fluid infusion device.

Also disclosed herein is a fluid infusion device to deliver medication fluid to a user. An exemplary embodiment of the fluid infusion device includes a housing having a reservoir cavity defined therein to receive a fluid reservoir containing the medication fluid. The device also includes a reservoir lock structure integrated with or coupled to the housing, wherein the reservoir lock structure mates with a compatible housing lock structure of the fluid reservoir to inhibit axial movement of the fluid reservoir relative to the housing. The device also includes a piston actuator having a distal end that includes a piston lock structure to removably couple the piston actuator to a piston of the fluid reservoir.

Also disclosed herein is an assembly for a fluid infusion device that delivers medication fluid to a user. An exemplary embodiment of the assembly includes a fluid reservoir and a compatible piston actuator. The fluid reservoir includes: a primary body section having a fluid chamber defined therein; a fitting section extending from the primary body section; a housing lock structure to removably couple the fluid reservoir to a housing of the fluid infusion device, the housing lock structure inhibiting axial movement of the fluid reservoir relative to the housing; and a piston located within the fluid chamber. The piston is axially movable relative to the primary body section, and the piston includes a tip section facing the fluid chamber and a base section opposite the tip section. The base section includes an actuator lock structure. The piston actuator controls axial movement of the piston within the fluid chamber. The piston actuator includes a piston lock structure that cooperates with the actuator lock structure of the piston to removably couple the piston to the piston actuator.

Another assembly for a fluid infusion device that delivers medication fluid to a user is also disclosed herein. An exemplary embodiment of the assembly includes a fluid reservoir and a compatible connector. The fluid reservoir includes a primary body section having a fluid chamber defined therein, a fitting section extending from the primary body section, and a plug component. The fitting section includes a sidewall structure having a through-hole formed therein, the sidewall structure at least partially defining a cavity. The plug component is rotatably coupled to the fitting section. The plug component includes a lower section located in the cavity, an actuator, and a fluid flow path defined in the lower section. The fluid flow path has an outlet opening and a chamber opening fluidly coupled to the fluid chamber. The sidewall structure blocks the outlet opening when the plug component is in a closed position, and the outlet opening is fluidly coupled to the through-hole when the plug component is in an open position. The connector includes a main body section having an interior to receive the fitting section of the fluid reservoir, an outlet port defined in the main body section, and a conduit extending from the main body section. The conduit is in fluid communication with the outlet port. The connector also includes an engagement structure to receive the actuator when the connector is coupled to the fluid reservoir. Rotation of the connector relative to the fluid reservoir from an unlocked position to a locked position causes the plug component to rotate within the cavity from the closed position to the open position. The outlet port is fluidly coupled to the through-hole when the connector is in the locked position, such that the conduit fluidly communicates with the fluid chamber, via the fluid flow path, when the connector is in the locked position.

Also disclosed herein is a fluid delivery assembly for a fluid infusion device that delivers medication fluid to a user. An exemplary embodiment of the fluid delivery assembly includes a primary body section, a fitting section, and a plug component. The primary body section has a fluid chamber defined therein. The fitting section extends from the primary body section, and it includes a sidewall structure having a through-hole formed therein. The sidewall structure at least partially defines a cavity. The plug component is rotatably coupled to the fitting section, and it has a lower section located in the cavity. The plug component also includes an actuator to facilitate rotation of the plug component, and a fluid flow path defined in the lower section. The fluid flow path has an outlet opening and a chamber opening fluidly coupled to the fluid chamber. The sidewall structure blocks the outlet opening when the plug component is in a closed position, and the outlet opening is fluidly coupled to the through-hole when the plug component is in an open position.

Another assembly for a fluid infusion device that delivers medication fluid to a user is also disclosed herein. An exemplary embodiment of the assembly includes a main body section having an interior to receive a fitting section of a fluid reservoir, an outlet port defined in the main body section, and a conduit extending from the main body section. The conduit is in fluid communication with the outlet port. The assembly also includes an engagement structure to receive an actuator of the fluid reservoir when the main body section is coupled to the fluid reservoir. Rotation of the main body section relative to the fluid reservoir from an unlocked position to a locked position results in rotation of the actuator from a closed position to an open position. The outlet port is fluidly coupled to the through-hole when the main body section is in the locked position.

Another assembly for a fluid infusion device that delivers medication fluid to a user is also disclosed herein. An exemplary embodiment of the assembly includes a fluid reservoir and a compatible connector. The fluid reservoir includes a primary body section having a fluid chamber defined therein, the fluid chamber terminating at a neck section. The reservoir also includes a septum that seals the neck section, the septum formed from a self-healing material. The reservoir has a fitting section extending from the primary body section, the fitting section having an outer wall surrounding the neck section. The reservoir also has a housing lock structure to removably couple the fluid reservoir to a housing of the fluid infusion device. The housing lock structure inhibits axial movement of the fluid reservoir relative to the housing. The connector includes a main body section, a hollow needle supported by the main body section, a conduit extending from the main body section and in fluid communication with the hollow needle, and a reservoir lock structure integrated with the main body section. The reservoir lock structure cooperates with a connector lock structure integrated with the outer wall of the fitting section. The hollow needle pierces the septum when the connector is coupled to the fitting section, and the hollow needle is held in fluid communication with the fluid chamber when the reservoir lock structure is engaged with the connector lock structure.

Another fluid delivery assembly for a fluid infusion device that delivers medication fluid to a user is also disclosed herein. The fluid delivery assembly includes: a primary body section having a fluid chamber defined therein, the fluid chamber terminating at a neck section; a septum that seals the neck section, the septum formed from a self-healing material; a fitting section extending from the primary body section, the fitting section having an outer wall surrounding the neck section; a connector lock structure integrated with the outer wall, the connector lock structure being compatible with a reservoir lock structure of an infusion set connector to removably secure the infusion set connector to the fitting section; and a housing lock structure to removably couple the fluid reservoir to a housing of the fluid infusion device. The housing lock structure inhibits axial movement of the fluid reservoir relative to the housing.

Another assembly for a fluid infusion device that delivers medication fluid to a user is also disclosed herein. An exemplary embodiment of the assembly includes a fluid reservoir and an infusion set component. The fluid reservoir includes: a primary body section having a fluid chamber defined therein; a housing lock structure to removably couple the fluid reservoir to a housing of the fluid infusion device, the housing lock structure inhibiting axial movement of the fluid reservoir relative to the housing; and a fitting section extending from the primary body section, the fitting section having a connector lock structure integrated therein. The infusion set component includes: tubing having a proximal end and a distal end; a cannula coupled to the distal end of the tubing; and a connector coupled to the proximal end of the tubing. The connector includes a main body section, a hollow needle supported by the main body section and in fluid communication with the tubing, and a reservoir lock structure integrated with the main body section. The reservoir lock structure cooperates with the connector lock structure of the fitting section to removably couple the connector to the fluid reservoir.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a plan view of an exemplary embodiment of a fluid delivery system that includes a fluid infusion device and an infusion set;

FIG. 2 is a cross-sectional view of an exemplary embodiment of a fluid reservoir;

FIG. 3 is a perspective view of an exemplary embodiment of a fluid infusion device and a fluid vial as arranged during an automated pump-fill operation;

FIG. 4 is a partial cross-sectional view of an exemplary embodiment of an assembly for a fluid infusion device;

FIG. 5 is a partial cross-sectional view of an exemplary embodiment of an assembly (including a piston and a piston actuator) for a fluid infusion device;

FIG. 6 is a side view of an exemplary embodiment of a fluid reservoir for a fluid infusion device;

FIG. 7 is a top view of the fluid reservoir shown in FIG. 6;

FIG. 8 is a side view of another exemplary embodiment of a fluid reservoir for a fluid infusion device;

FIG. 9 is a top view of the fluid reservoir shown in FIG. 8;

FIG. 10 is a top perspective view of an exemplary embodiment of a fluid reservoir that is compatible with a needleless infusion set connector;

FIG. 11 is a top perspective view of an exemplary embodiment of a plug component suitable for use with the fluid reservoir shown in FIG. 10;

FIG. 12 is an axial cross-sectional view of the plug component shown in FIG. 11;

FIG. 13 is a top perspective view of a portion of a fluid infusion device that is compatible with the fluid reservoir shown in FIG. 10;

FIG. 14 is a top view of the portion of the fluid infusion device shown in FIG. 13;

FIG. 15 is a top perspective view of the fluid reservoir (shown in FIG. 10) installed in the fluid infusion device;

FIG. 16 is a top view of the fluid reservoir (shown in FIG. 10) installed in the fluid infusion device;

FIG. 17 is a top perspective view of an exemplary embodiment of a connector suitable for use with a needleless infusion set;

FIG. 18 is a top view of the connector shown in FIG. 17;

FIG. 19 is a bottom perspective view of the connector shown in FIG. 17;

FIG. 20 is a cross-sectional view of the connector from the perspective of line 20-20 in FIG. 18;

FIG. 21 is a top view of the connector coupled to the fluid reservoir in an unlocked state;

FIG. 22 is a top view of the connector coupled to the fluid reservoir in a locked state;

FIG. 23 is a top perspective view of the connector coupled to the fluid reservoir in a locked state;

FIG. 24 is a cross-sectional view of the connector and a portion of the fluid reservoir, from the perspective of line 24-24 in FIG. 22;

FIG. 25 is a top view of an exemplary embodiment of a fluid reservoir suitable for use with a fluid infusion device;

FIG. 26 is a partial cross-sectional view of the fluid reservoir shown in FIG. 25, with a needled infusion set connector coupled thereto; and

FIG. 27 is a top view of the assembly shown in FIG. 26.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

Certain terminology may be used in the following description for the purpose of reference only, and thus are not intended to be limiting. For example, terms such as “upper”, “lower”, “above”, and “below” refer to directions in the drawings to which reference is made. Terms such as “front”, “back”, “rear”, “side”, “outboard,” and “inboard” describe the orientation and/or location of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

The subject matter described here relates to certain assemblies, components, and features of a fluid infusion system of the type used to treat a medical condition of a patient. The fluid infusion system is used for infusing a medication fluid into the body of a user. The non-limiting examples described below relate to a medical device used to treat diabetes (more specifically, an insulin pump), although embodiments of the disclosed subject matter are not so limited. Accordingly, the medication fluid is insulin in certain embodiments. In alternative embodiments, however, many other fluids may be administered through infusion such as, but not limited to, disease treatments, drugs to treat pulmonary hypertension, iron chelation drugs, pain medications, anti-cancer treatments, medications, vitamins, hormones, or the like.

For the sake of brevity, conventional features and functionality related to infusion systems, insulin pumps, infusion sets, and fluid reservoirs may not be described in detail here. Examples of infusion pumps and/or related pump drive systems used to administer insulin and other medications may be of the type described in, but not limited to, U.S. Pat. Nos. 5,505,709; 6,485,465; 6,554,798; 6,558,351; 6,659,980; 6,752,787; 6,817,990; 6,932,584; and 7,621,893; which are herein incorporated by reference.

Fluid Infusion System

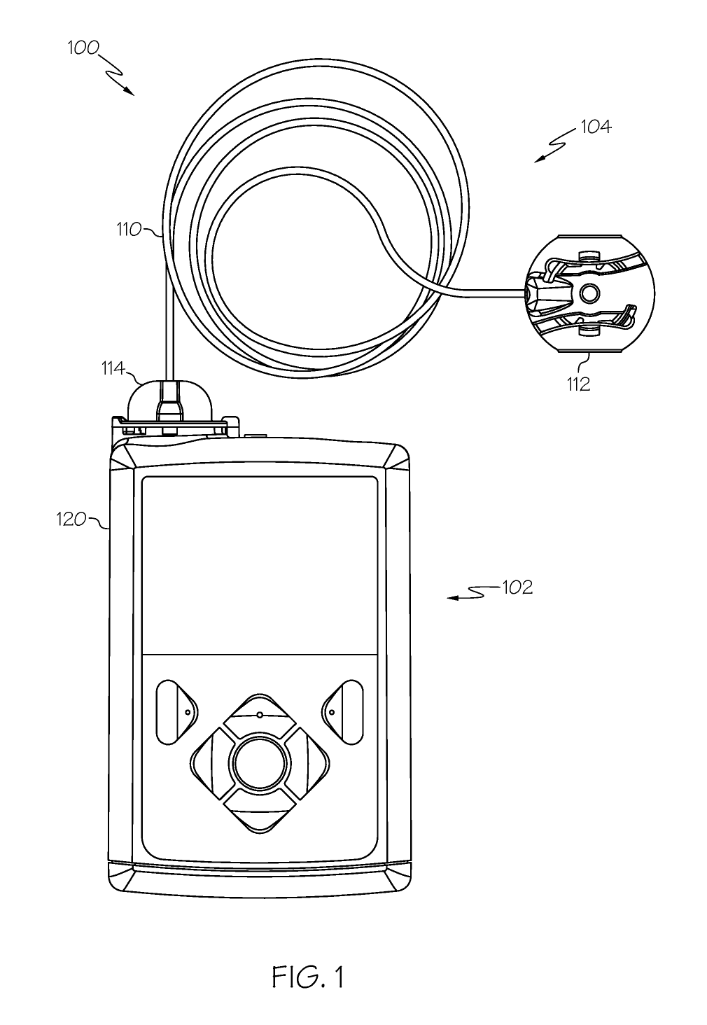

Referring to the drawings, FIG. 1 is a plan view of an exemplary embodiment of a fluid delivery system 100 that includes a portable fluid infusion device 102 and a fluid conduit assembly that takes the form of an infusion set 104. For this particular embodiment, the infusion set 104 can be coupled to the fluid infusion device 102 as depicted in FIG. 1. The fluid infusion device 102 accommodates a fluid reservoir (hidden from view in FIG. 1) for the medication fluid to be delivered to the user.

The illustrated embodiment of the infusion set 104 includes, without limitation: a length of tubing 110; an infusion unit 112 coupled to the distal end of the tubing 110; and a connector 114 coupled to the proximal end of the tubing 110. The fluid infusion device 102 is designed to be carried or worn by the patient, and the infusion set 104 terminates at the infusion unit 112 such that the fluid infusion device 102 can deliver fluid to the body of the patient via the tubing 110. The infusion unit 112 includes a cannula (hidden from view in FIG. 1) that is coupled to the distal end of the tubing 110. The cannula is inserted into the skin and is held in place during use of the fluid infusion device 102.

The infusion set 104 defines a fluid flow path that fluidly couples the fluid reservoir to the infusion unit 112. The connector 114 mates with and couples to a section of the fluid reservoir, which in turn is coupled to a housing 120 of the fluid infusion device 102. The connector 114 establishes the fluid path from the fluid reservoir to the tubing 110. Actuation of the fluid infusion device 102 causes the medication fluid to be expelled from the fluid reservoir, through the infusion set 104, and into the body of the patient via the infusion unit 112 and cannula at the distal end of the tubing 110. Accordingly, when the connector 114 is installed as depicted in FIG. 1, the tubing 110 extends from the fluid infusion device 102 to the infusion unit 112, which in turn provides a fluid pathway to the body of the patient.

Locking Interface Between Piston and Actuator

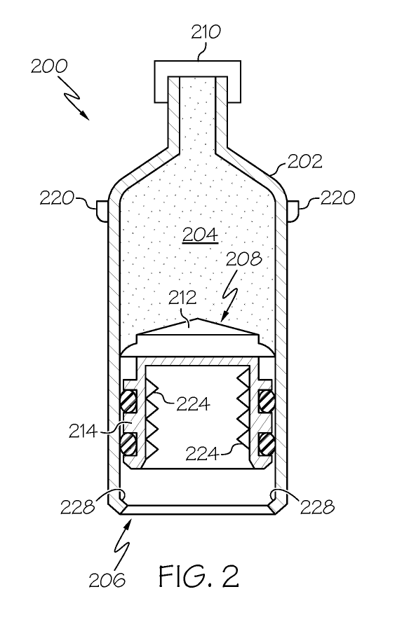

FIG. 2 is a schematic cross-sectional view of an exemplary embodiment of a fluid reservoir 200. The fluid reservoir 200 is shaped, sized, and configured for compatibility with a fluid infusion device (e.g., the fluid infusion device 102) that delivers medication fluid to a user. For the illustrated embodiment, the fluid reservoir 200 is designed to fit inside a reservoir cavity defined in the housing of the fluid infusion device. Thus, the reservoir cavity receives the fluid reservoir 200, which is held in position during operation of the fluid infusion device. The fluid reservoir 200 generally includes a primary body section 202 having a fluid chamber 204 defined therein. The base 206 of the primary body section 202 is open to accommodate insertion of a piston 208 into the fluid chamber 204, and to accommodate actuation of the piston 208 during operation of the fluid infusion device. The fluid reservoir 200 includes a fitting section 210 that extends from the primary body section 202. The fitting section 210 is schematically represented in a simplified form in FIG. 2. It should be appreciated that the fitting section 210 can be shaped, sized, and configured as needed to accommodate the particular application and embodiment.

The piston 208 is located within the fluid chamber 204, and it is axially movable relative to the primary body section 202 (up and down as oriented in FIG. 2). The piston 208 includes a tip section 212 facing the fluid chamber 204, and a base section 214 opposite the tip section 212. The fluid infusion device includes an actuator (not shown in FIG. 2) that engages the base section 214 to cause the piston 208 to move in a controlled and regulated manner. In this regard, the axial position of the actuator is controlled to move the piston 208 toward the fitting section 210 during fluid delivery operations, and is controlled to move the piston 208 in both directions during pump-fill operations.

The illustrated embodiment of the fluid reservoir 200 includes a housing lock structure 220, which is suitably configured to removably couple the fluid reservoir 200 to the housing of the fluid infusion device. The housing lock structure 220 is schematically depicted in a simplified form in FIG. 2. In certain embodiments, the housing lock structure 220 can be realized as any of the following, without limitation: a threaded structure (e.g., external threads as shown or internal threads); a key or keyway structure; a snap-fitting structure; a bayonet mount structure; a clip mechanism; or the like. The housing lock structure 220 mates with and cooperates with a corresponding reservoir lock structure (not shown in FIG. 2) of the housing to form a releasable locking engagement. For example, the housing can include a threaded feature that mates with a threaded structure located on the primary body section 202 of the fluid reservoir 200. When the housing lock structure 220 is coupled and secured to the reservoir lock structure, the cooperating lock structures inhibit axial movement of the fluid reservoir 200 relative to the housing. Maintaining the fluid reservoir 200 in a stationary position is important to ensure accurate and precise delivery of medication fluid, and to accommodate the pump-fill methodology described herein.

The illustrated embodiment of the piston 208 includes an actuator lock structure integrated with, formed on, or coupled to the base section 214. The actuator lock structure is suitably configured to removably couple the piston 208 to a piston actuator (not shown in FIG. 2) of the fluid infusion device. FIG. 2 depicts one exemplary embodiment where the actuator lock structure is realized as a threaded structure 224 (e.g., internal threads as shown or external threads) that mates with a threaded distal end of the piston actuator. In alternative embodiments, the actuator lock structure can be realized as any of the following, without limitation: a key or keyway structure; a snap-fitting structure; a bayonet mount structure; a clip mechanism; or the like. The actuator lock structure 224 mates with and cooperates with the end of the actuator (not shown in FIG. 2) to form a releasable locking engagement. When the actuator lock structure 224 is coupled and secured to the actuator, the locking engagement inhibits movement of the piston 208 relative to the actuator, and allows the actuator to accurately control the position of the piston 208 during reservoir filling, fluid path priming, and fluid delivery operations.

The fluid reservoir 200 also includes a retaining structure 228 formed at the base end of the primary body section 202. The retaining structure 228 is suitably configured to retain the piston 208 inside the primary body section 202. In other words, the retaining structure 228 inhibits removal of the piston 208 from the fluid chamber 204. Depending on the particular embodiment, the retaining structure 228 can be realized as any of the following, without limitation: an inwardly protruding collar (as depicted in FIG. 2); a number of inwardly protruding tabs; a spring clip; a threaded ring; or the like. The retaining structure 228 can be fabricated (e.g., by swaging, heat-staking, or the like) during the assembly process after the piston 208 has been inserted into the primary body section 202. The retaining structure 228 is desirable to prevent accidental removal of the piston 208 during automated reservoir filling operations. An additional purpose of the retaining structure 228 could be to prevent free rotation of the piston 208 when it is in contact with the retaining structure 288. For example, the retaining structure 288 can be provided with teeth or features that mate with the piston 208. This additional feature will ensure that the user is able to twist and unscrew a threaded piston 208 without the piston 208 rotating freely in the primary body section 202 of the fluid reservoir 200.

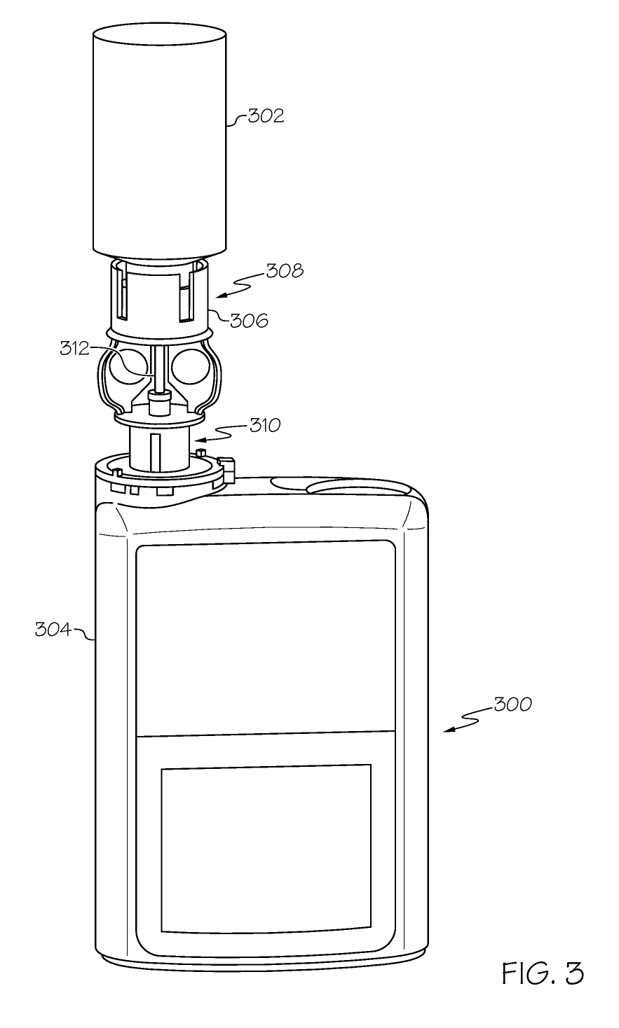

As mentioned above, a fluid infusion device and a compatible fluid reservoir can support an automated reservoir filling procedure that utilizes the fluid infusion device as the filling mechanism. In this regard, FIG. 3 is a perspective view of an exemplary embodiment of a fluid infusion device 300 and a vial 302 as arranged during an automated pump-fill operation. A fluid reservoir (which is primarily hidden from view in FIG. 3) is installed into the reservoir cavity of the fluid infusion device 300, and is locked into position such that the primary body section of the fluid reservoir is stationary relative to the housing 304 of the fluid infusion device 300.

The arrangement shown in FIG. 3 includes an interface component 306 that physically and fluidly couples the vial 302 to the fluid reservoir. In certain embodiments, the interface component 306 is physically coupled to the housing 304 (in lieu of, or in addition to, the fluid reservoir). A first end 308 of the interface component 306 attaches to the vial 302, and a second end 310 of the interface component 306 attaches to the fluid reservoir. The interface component 306 includes a hollow needle 312 having one end that pierces a septum of the vial 302 when the interface component 306 is secured to the vial, and having a second end that pierces a septum of the fluid reservoir when the interface component 306 is secured to the fluid reservoir. When the vial 302 is connected to the fluid infusion device 300 as shown in FIG. 3, the fluid infusion device 300 can be operated to actuate the piston of the fluid reservoir (see FIG. 2) in a controlled manner to draw medication fluid from the vial 302 into the fluid reservoir. The automated filling process can be controlled in an appropriate manner that inhibits or reduces the amount of air bubbles introduced into the fluid reservoir, and/or in a manner that removes or ejects air bubbles from the fluid reservoir.

When filling the fluid reservoir, the piston is moved backward (toward the base of the reservoir) to accommodate the medication fluid. The piston can also be moved forward if needed to initiate the filling process, to perform pulsation during the filling procedure, to prime the fluid flow path, or the like. The piston can also be moved forward for other reasons: to pressurize the vial before fluid transfer, or to transfer air bubbles from the fluid reservoir into the medication vial. To facilitate accurate positioning of the piston during backward movements, the piston is temporarily locked with the actuator of the fluid infusion device.

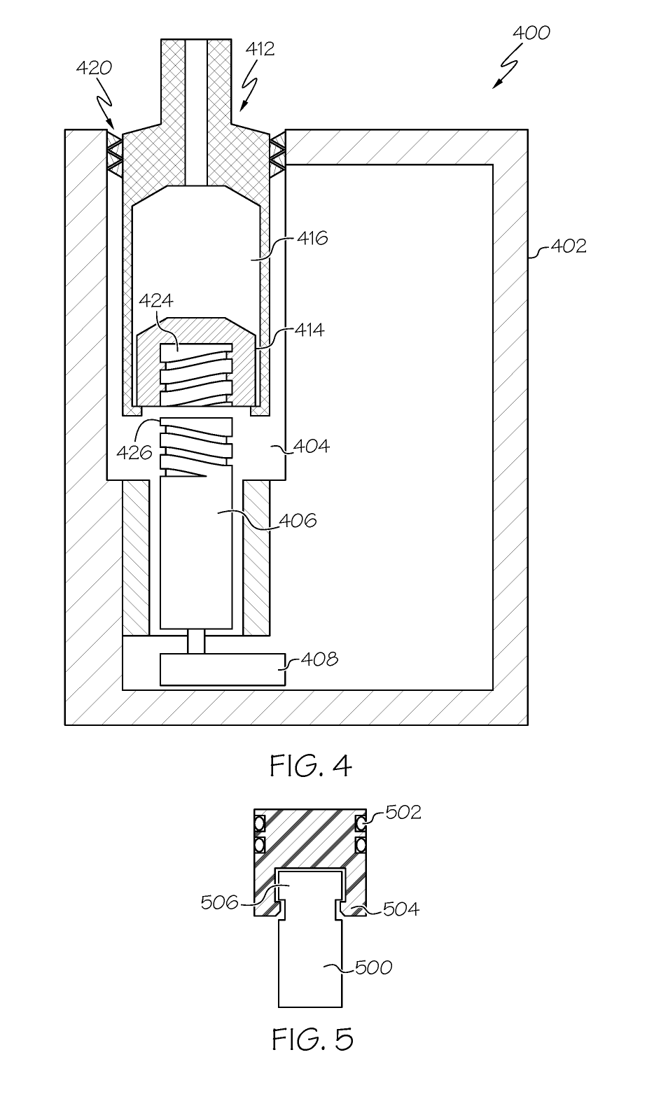

FIG. 4 is a partial cross-sectional view of an exemplary embodiment of an assembly for a fluid infusion device 400. FIG. 4 is overly simplified for the sake of clarity, and various features, components, and structures found in conventional infusion devices are not shown or described herein. The fluid infusion device 400 generally includes a housing 402, a reservoir cavity 404 defined within the housing 402, a piston actuator 406 that moves within the reservoir cavity 404, and a drive motor 408 coupled to the actuator 406. The fluid infusion device 400 includes or cooperates with a fluid reservoir 412, which may be configured as described previously. In this regard, the fluid reservoir 412 includes a movable piston 414 that resides within a fluid chamber 416. For ease of illustration, FIG. 4 shows the piston 414 decoupled from the actuator 406.

The fluid infusion device 400 includes a reservoir lock structure that is integrated with, or coupled to, the housing 402. The reservoir lock structure mates with a compatible housing lock structure of the fluid reservoir 412, as described above. The embodiment depicted in FIG. 4 employs a threaded engagement 420 to lock the fluid reservoir 412 in the housing 402. The threaded engagement 420 includes cooperating structures or features on the fluid reservoir 412 and the housing 402 that allow the user to screw the fluid reservoir 412 into the housing 402 until it locks into position. As explained above, however, the fluid reservoir 412 and the housing 402 can be alternatively configured with other locking structures, devices, features, or components (other than a threaded lock structure).

The illustrated version of the piston 414 includes an internally threaded cavity 424. This threaded structure of the piston 414 represents an exemplary embodiment of an actuator lock structure that mates with a piston lock structure of the piston actuator 406. More specifically, the internally threaded cavity 424 cooperates with an externally threaded tip 426 at the distal end of the piston actuator 406. In other words, the piston lock structure shown in FIG. 4 is realized as external locking threads formed in the distal end of the piston actuator 406. The threaded engagement facilitates removable coupling of the piston 414 to the actuator 406.

In accordance with certain embodiments, the thread pitch of the threaded engagement between the fluid reservoir 412 and the housing 402 is different than the thread pitch of the threaded engagement between the piston 414 and the actuator 406. The variation in thread pitch facilitates coupling of the actuator 406 to the piston 414 in response to coupling the fluid reservoir 412 to the housing 402. For example, if one full rotation (360 degrees) is required to install and lock the fluid reservoir 412 into the housing 402, then the thread pitch of the actuator-piston engagement can be configured such that less than one full rotation (e.g., only 180 degrees or only 90 degrees) is needed to secure the actuator 406 to the piston 414. Thus, installation and locking of the fluid reservoir 412 into the housing 402 also functions to secure and lock the piston 414 to the actuator 406. In accordance with an exemplary installation procedure, the actuator 406 is initially extended toward the opening of the reservoir cavity 404 to ensure that the externally threaded tip 426 mates with the internally threaded cavity 424 when the fluid reservoir 412 is introduced into the housing 402. In accordance with another exemplary installation procedure, the actuator 406 can be activated to ensure proper seating and coupling with the piston 414 before the medication fluid is drawn into the fluid chamber 416.

The motor 408 is controlled in an appropriate manner to cause the piston actuator 406 to move in the axial direction (up and down in FIG. 4). In certain embodiments, the motor 408 rotates a screw drive component, which in turn causes the actuator 406 to slide up or down within the reservoir cavity 404. Thus, the motor 408 drives the actuator 406 to control axial movement of the piston 414 within the fluid chamber 416 as needed. The locking threads of the piston 414 and the actuator 406 allow retraction of the piston 414 within the fluid chamber 416 during the automated filling procedure.

FIG. 5 is a partial cross-sectional view of another exemplary embodiment of an assembly for a fluid infusion device. FIG. 5 shows the distal end of a piston actuator 500 and a compatible piston 502 of a fluid reservoir. The actuator lock structure of the piston 502 includes a snap-fitting receptacle 504 having resilient hooks, arms, or a rim that mates with a compatible snap-fitting neck structure 506 on the distal end of the piston actuator 500. The neck structure 506 can include a plurality of detents that receive the locking features of the snap-fitting receptacle 504, or it can include a single groove to receive the locking features.

In alternative embodiments, the actuator lock structure of the piston 502 includes a snap-fitting neck structure that mates with a compatible snap-fitting receptacle on the distal end of the piston actuator 500. In other embodiments that employ a threaded engagement between the piston and the actuator, the piston can include an externally threaded neck structure extending from its base section, and the actuator can include an internally threaded cavity that receives and couples with the threaded neck structure. These and other configurations are contemplated by this disclosure.

Regardless of the manner in which it is implemented, the locking arrangement between the piston and the piston actuator is suitably designed and fabricated to provide adequate locking force to support automated filling and fluid delivery operations. Moreover, the locking arrangement is suitably designed and fabricated to allow removal of the fluid reservoir if so desired. For example, it may be desirable to replace the fluid reservoir on a daily basis, after the medication fluid is depleted, once a week, or the like. Accordingly, the locking force/tension between the piston and the actuator should be strong enough to support normal operation of the fluid infusion device, yet weak enough to accommodate removal and replacement of the fluid reservoir.

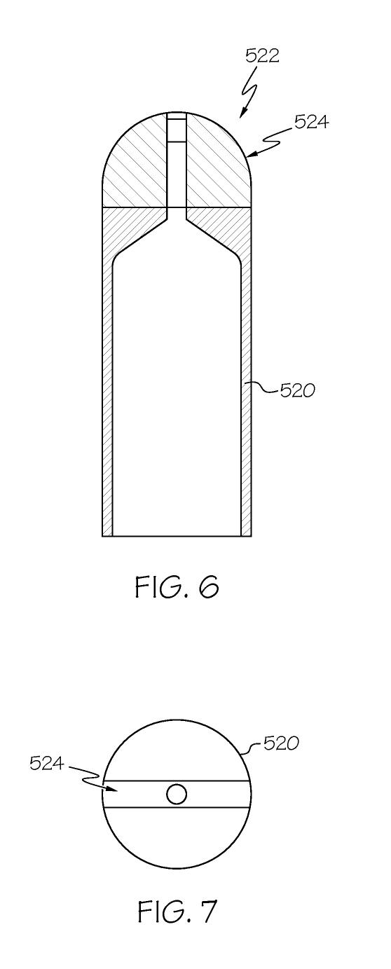

A fluid reservoir of the type described herein can also include features that improve handling, purchase, and grip during installation. In this regard, FIG. 6 is a side view of an exemplary embodiment of a fluid reservoir 520 for a fluid infusion device, and FIG. 7 is a top view of the fluid reservoir 520. The fitting section 522 of the fluid reservoir 520 includes at least one finger wing 524 that extends upward. As shown in FIG. 7, the finger wing 524 can be arranged across the diameter of the fluid reservoir 520 to provide leverage when twisting the fluid reservoir 520 into and out of the housing of the fluid infusion device.

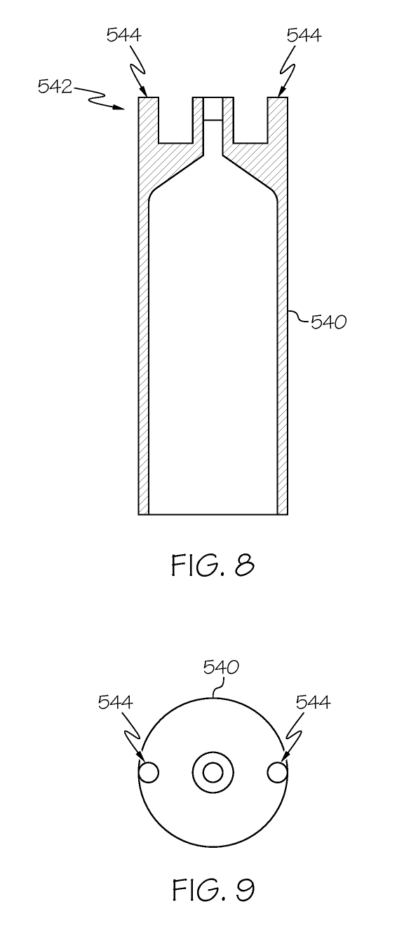

FIG. 8 is a side view of another exemplary embodiment of a fluid reservoir 540, and FIG. 9 is a top view of the fluid reservoir 540. The fitting section 542 of the fluid reservoir 540 includes a plurality of finger posts 544 that extend upward. The finger posts 544 can be arranged at or near the perimeter of the fluid reservoir 540, as depicted in FIG. 9. The finger posts 544 can be suitably configured and arranged to provide leverage when twisting the fluid reservoir 540 into and out of the housing of the fluid infusion device.

Needleless Infusion Set Connector

In accordance with certain embodiments of the fluid infusion system, the fluid reservoir and the infusion set component can be cooperatively configured as a needleless assembly. Moreover, the fluid reservoir can be suitably configured to support the automatic pump-fill features described in the preceding section of this disclosure. In this regard, the fluid reservoir can be installed and secured to the housing of the fluid infusion device, and filled using an appropriate pump-fill methodology. Thereafter, a needleless infusion set connector can be coupled and secured to the fitting section of the fluid reservoir (and/or to a section of the housing) to establish the fluid flow path from the reservoir to the fluid delivery tube or conduit of the infusion set.

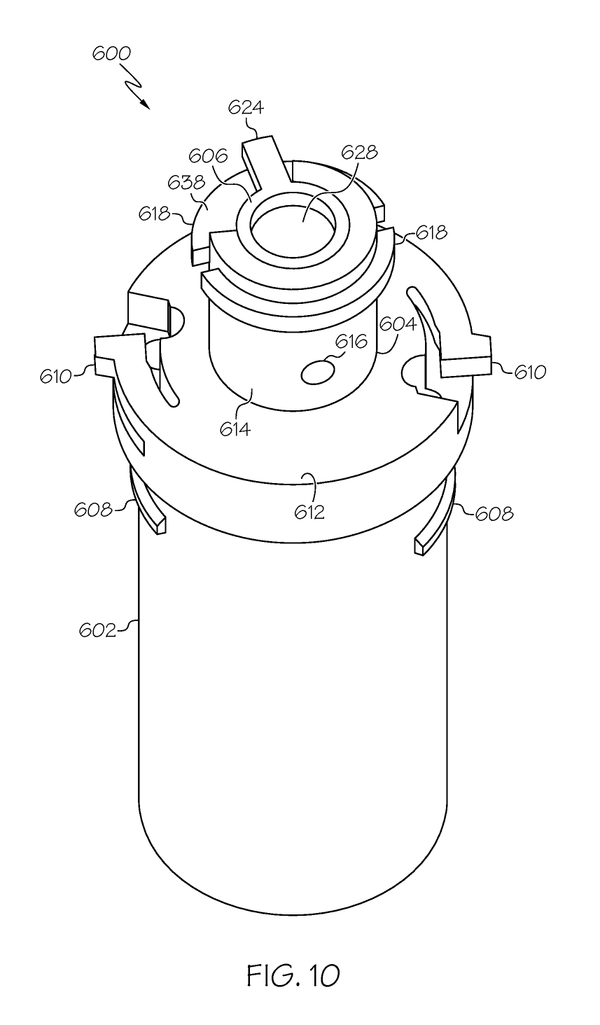

FIG. 10 is a top perspective view of an exemplary embodiment of a fluid reservoir 600 that is compatible with a needleless infusion set connector. The fluid reservoir 600 shares some of the features, structures, and functionality of the fluid reservoirs described in more detail above, and common aspects will not be redundantly described in this section. The fluid reservoir 600 generally includes, without limitation: a primary body section 602 having a fluid chamber defined therein; a fitting section 604 extending from the primary body section 602; and a plug component 606 rotatably coupled to the fitting section 604. Although hidden from view in FIG. 10, the fluid reservoir 600 includes a piston inside the primary body section 602, wherein the piston can be actuated by the fluid infusion device as needed.

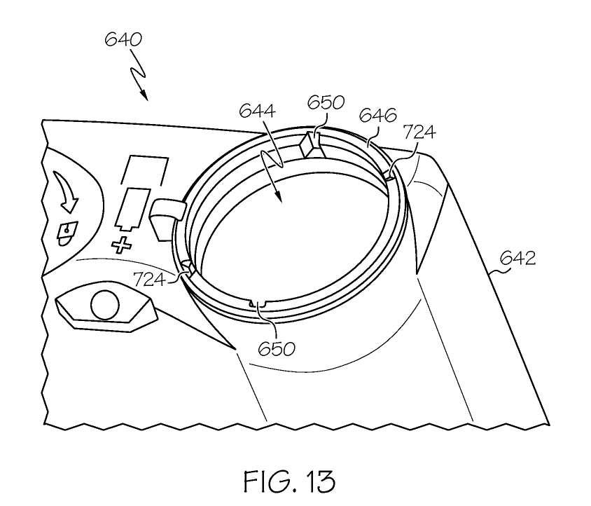

The illustrated embodiment of the fluid reservoir 600 includes at least one housing lock structure to accommodate installation and removable coupling to the housing of the fluid infusion device. For example, the fluid reservoir 600 includes a threaded structure 608 that mates with corresponding structure of the housing of the fluid infusion device. The fluid reservoir 600 also includes two locking arms 610 that engage corresponding structure of the housing to secure the fluid reservoir 600 to the housing and to prevent unintentional removal of the fluid reservoir 600. Operation of the locking arms 610 is described in more detail below with reference to FIGS. 13-16.

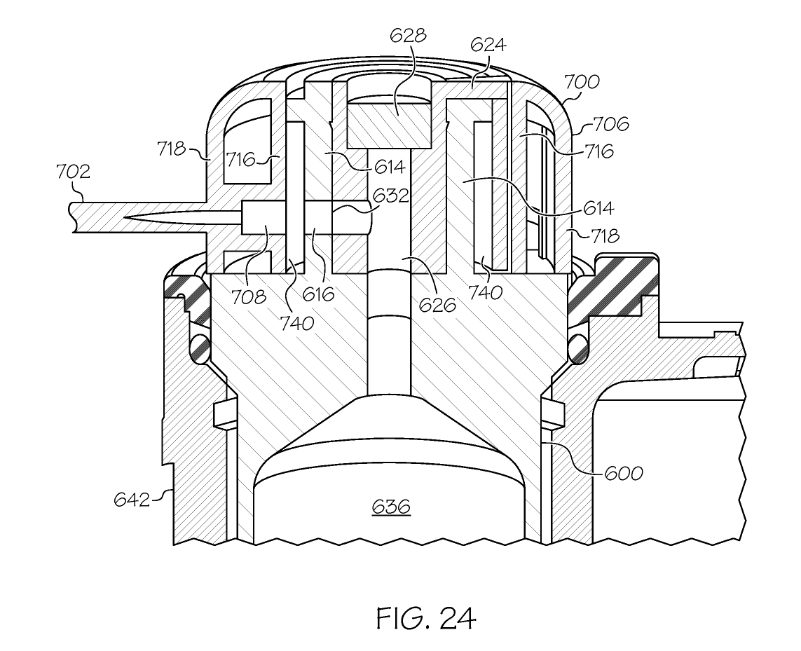

The fitting section 604 of the fluid reservoir 600 extends above a top section 612. For this particular embodiment, the locking arms 610 are integrally formed in the top section 612. The fitting section 604 includes a sidewall structure 614, which can be an annular structure as depicted in FIG. 10. The sidewall structure 614 includes a through-hole 616 formed therein. FIG. 10 shows the external opening of the through-hole 616; the internal opening (which is hidden from view) is fluidly coupled to an interior cavity of the fitting section 604. The interior cavity is at least partially defined by the sidewall structure 614 (see FIG. 24). As explained in more detail below, the through-hole 616 represents a portion of the fluid delivery pathway from the fluid chamber of the reservoir 600 to the body of the patient.

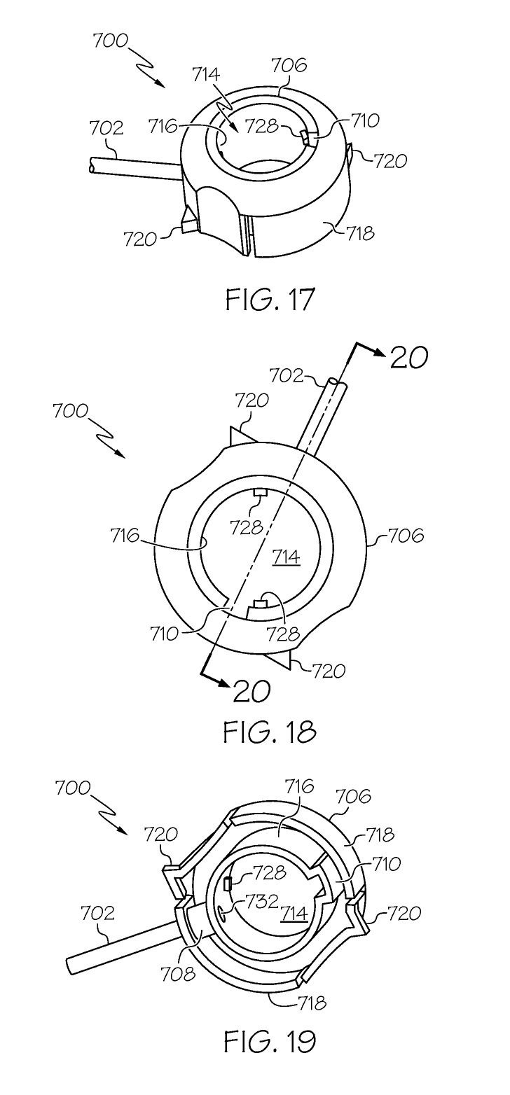

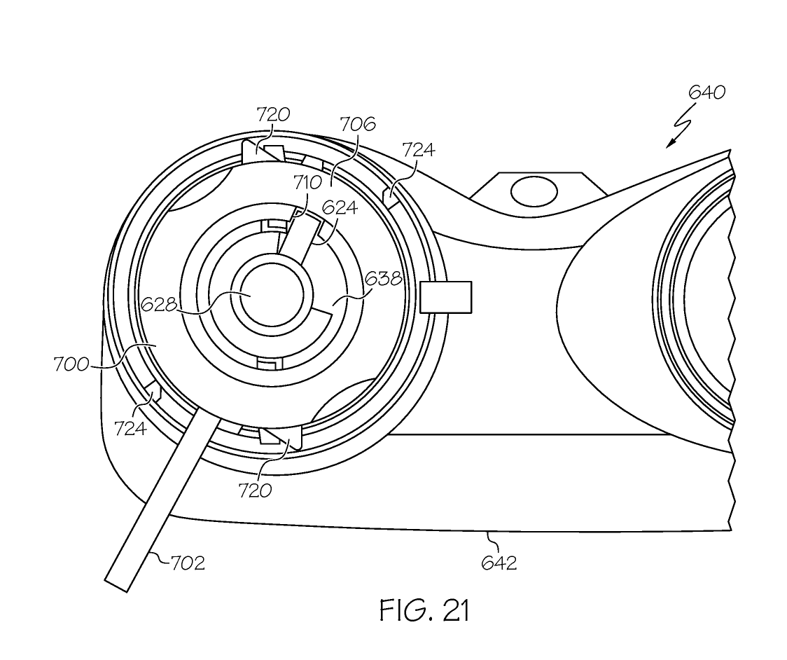

The end of the fitting section 604 includes or cooperates with coupling structure 618 that is compatible with similar features or structure of the connector (see FIGS. 17-20). For this particular embodiment, the coupling structure 618 is realized as a slotted rim that receives corresponding tabs, prongs, or arms of the connector. The coupling structure 618 facilitates installation and locking of the connector to the fitting section 604 by rotating the connector relative to the fitting section 604. As explained below with reference to FIGS. 21-24, rotation of the connector serves two purposes: (1) to secure the connector onto the fluid reservoir 600 and/or onto the housing of the fluid infusion device; and (2) to actuate and rotate the plug component 606, which establishes a fluid path between the reservoir 600 and the tube of the infusion set component.

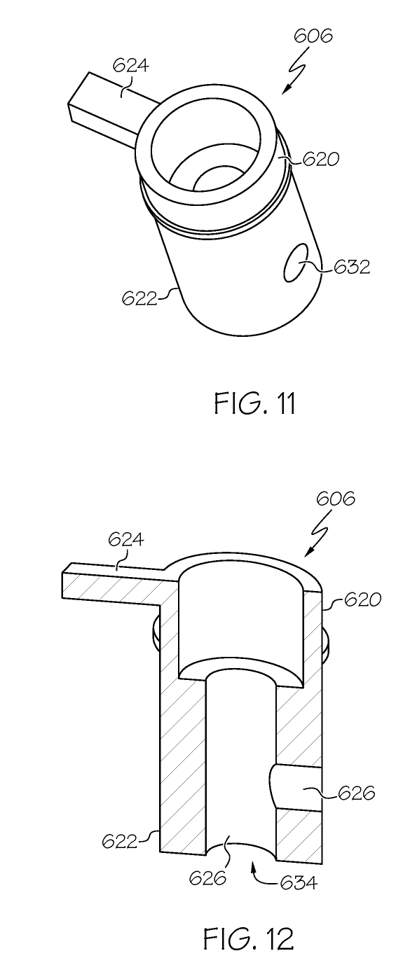

The plug component 606 is shown by itself in FIG. 11 and FIG. 12. The plug component 606 includes an upper section 620, a lower section 622, an actuator 624, a fluid flow path 626 defined in the lower section 622, and a septum 628 in the upper section 620. FIG. 10 shows the septum 628 in place, but FIGS. 11 and 12 do not include the septum 628 (for simplicity and ease of illustration). The septum 628 is positioned within the upper section 620 to form a fluid tight seal that inhibits leakage from the fluid flow path. The septum 628 can be press-fit into the upper section 620 or affixed using a suitable adhesive. The septum 628 is formed from a self-healing material (e.g., silicone) that accommodates piercing by a hollow needle to fill the fluid chamber with medication fluid. As explained in more detail in the preceding section, a transfer guard or other type of fluid interface component can include a hollow needle that accommodates transfer of the medication fluid from a vial to the fluid reservoir 600 during an automatic pump-fill procedure. After the fluid reservoir 600 is filled, the hollow needle is extracted from the septum 628, which re-seals itself to maintain the fluid tight seal.

Referring to FIG. 11 and FIG. 12, the fluid flow path 626 includes an outlet opening 632 and a chamber opening 634. The medication fluid is delivered through the outlet opening 632 during fluid delivery cycles of the infusion device. The chamber opening 634 is fluidly coupled to the fluid chamber 636 of the reservoir 600 (see FIG. 24) to allow the medication fluid to pass into the chamber (during filling operations) and to pass out of the chamber (during fluid delivery cycles). Although the specific shape, orientation, and layout of the fluid flow path 626 can vary from one embodiment to another, the illustrated plug component 606 employs a fluid flow path 626 that transitions in a T-shaped manner such that the outlet opening 632 can be aligned with the through-hole 616 when the plug component 606 is manipulated into its open position (as depicted in FIG. 10 and FIG. 24, for example).

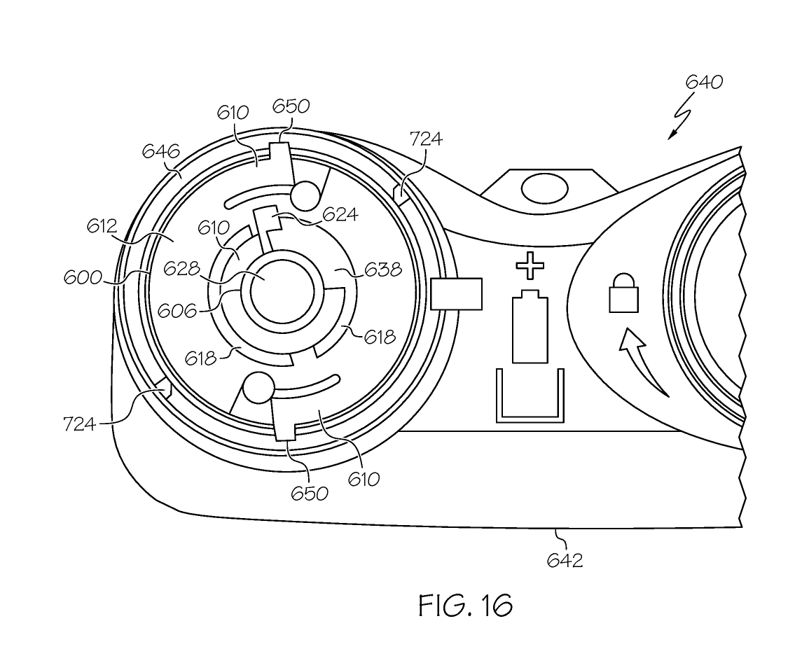

The plug component 606 can rotate within the cavity of the fitting section 604 between a closed position and an open position. The actuator 624 can be manipulated as needed to transition the plug component 606 from the closed and open positions as needed. FIGS. 10, 15, and 22-24 depict the plug component 606 in the open position, and FIGS. 16 and 21 depict the plug component 606 in the closed position. The fitting section 604 can be suitably configured with structure or features that limit rotation of the plug component 606 relative to the fitting section 604. For example, the fitting section 604 can include a guide 638 that prevents over-rotation of the plug component 606. The guide 638 defines two endpoints for the actuator 624, wherein the endpoints correspond to the closed position and the open position.

When the plug component 606 is in the closed position (or in any position other than the open position), the outlet opening 632 of the fluid flow path 626 is misaligned with the through-hole 616 formed in the sidewall structure 614. Consequently, the sidewall structure 614 blocks the outlet opening when the plug component 606 is in the closed position, which seals the fluid flow path 626 and, therefore, seals the fluid chamber 636. In practice, the plug component 606 and/or the fitting section 604 can include a suitably configured sealing element (such as a small o-ring) that enhances the fluid tight seal formed between the outlet opening 632 and the sidewall structure 614. The fluid reservoir 600 can be provided to the user with the plug component 606 in the closed position to facilitate filling of the fluid chamber 636 with the medication fluid.

When the plug component 606 is in the open position, the outlet opening 632 of the fluid flow path 626 is fluidly coupled to the through-hole 616 (see FIG. 24). As explained in more detail below, moving the plug component 606 into the open position creates a continuous delivery pathway for the medication fluid from the fluid chamber 636, through the fluid flow path 626, and into the through-hole 616.

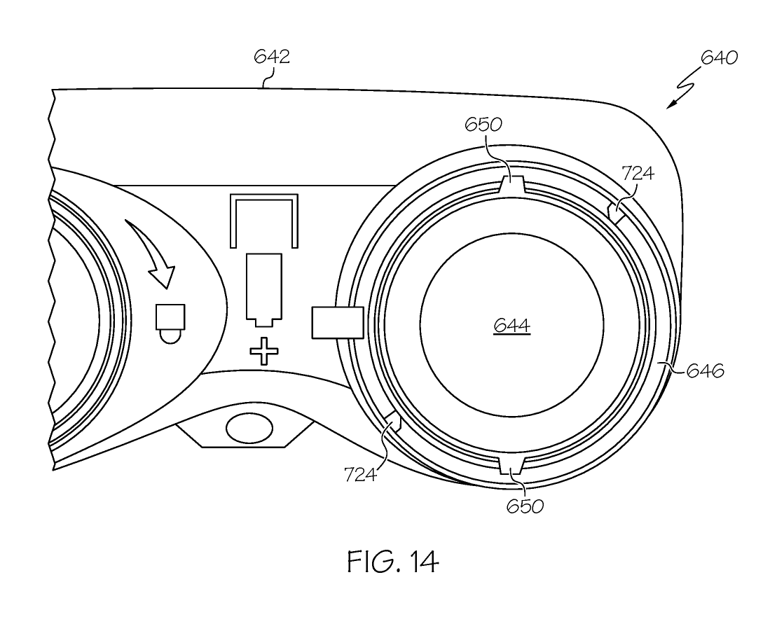

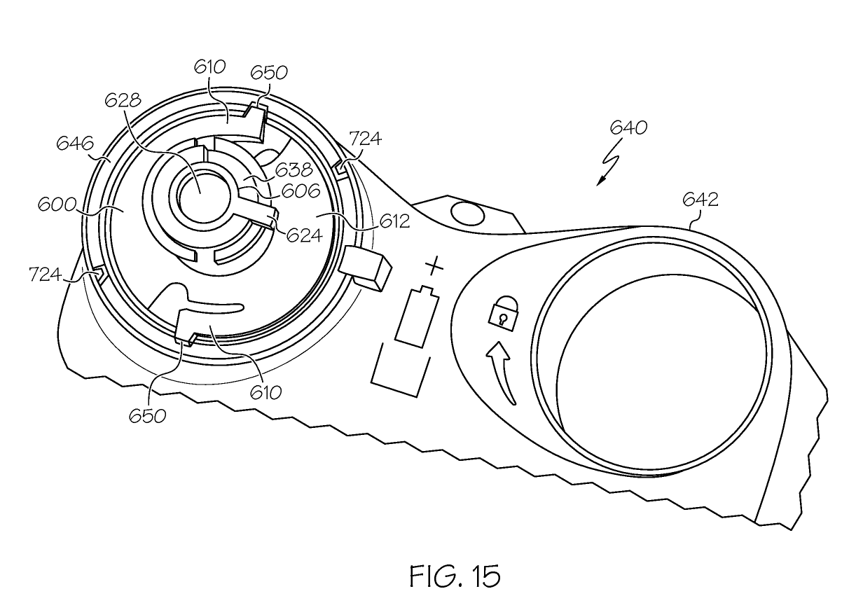

FIG. 13 is a top perspective view of a portion of a fluid infusion device 640 that is compatible with the fluid reservoir 600, and FIG. 14 is a top view of the portion of the fluid infusion device 640. FIG. 13 and FIG. 14 depict the housing 642 of the fluid infusion device 640 without the fluid reservoir 600 installed therein. The housing 642 includes a reservoir cavity 644 defined therein. The illustrated embodiment includes a collar 646, which may be integrated into the housing 642 or implemented as a distinct component that is coupled to the housing 642 at or near the opening of the reservoir cavity 644. For the embodiment described here, the housing 642 includes a threaded feature that serves as a reservoir lock structure. More specifically, the reservoir cavity 644 can include a threaded opening that allows the fluid reservoir 600 to be screwed into the housing 642. The collar 646 cooperates with the locking arms 610 of the fluid reservoir 600 to lock the reservoir 600 in place after it has been screwed into place. In this regard, the collar 646 can include detents 650 that are shaped, sized, and located to mate with the ends of the locking arms 610. FIG. 15 and FIG. 16 depict the fluid reservoir 600 installed in its locked position in the fluid infusion device 640. The tips of the locking arms 610 snap into the detents 650 to lock the fluid reservoir 600 in place. The user can squeeze the locking arms 610 inward to disengage them from the detents 650, which allows the fluid reservoir 600 to be removed from the housing 642.

In some embodiments, the fluid reservoir 600 can be filled in a traditional manner (e.g., manually or by a filling station) by piercing the septum 628 with a hollow needle that is in fluid communication with the source of medication fluid. In accordance with the exemplary embodiment described here, however, the fluid infusion device is operated in a pump-fill mode to fill the fluid reservoir 600 (see FIG. 3 and the related description). After the reservoir 600 has been filled and otherwise prepared for use, a compatible needleless infusion set connector can be attached to establish the fluid flow path from the reservoir 600 to the infusion site.

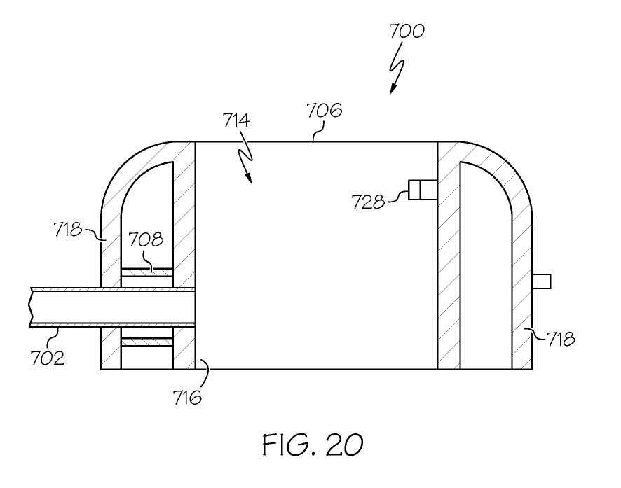

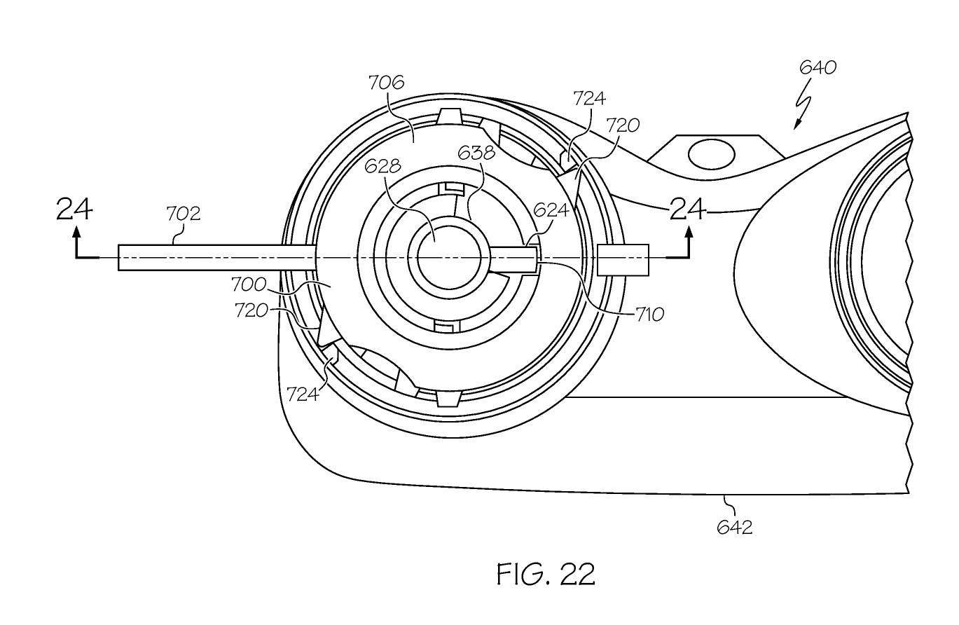

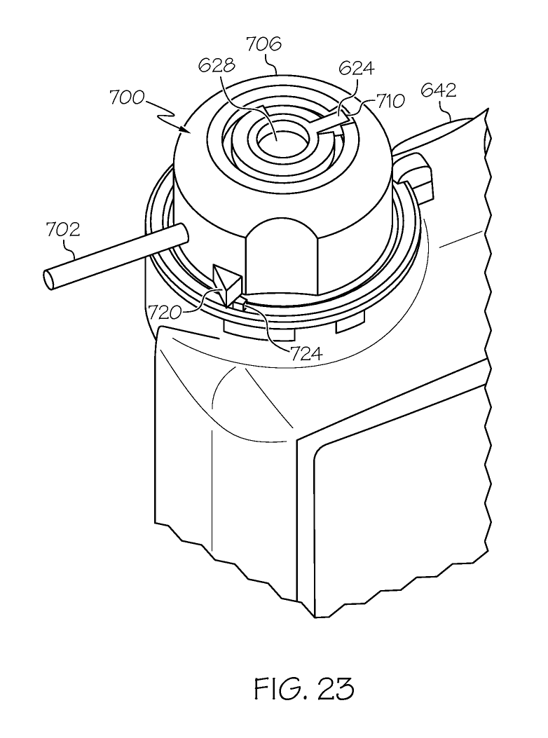

An exemplary embodiment of a needleless infusion set connector 700 will be described with reference to FIGS. 17-24. FIG. 17 is a top perspective view of the connector 700, FIG. 18 is a top view of the connector 700, FIG. 19 is a bottom perspective view of the connector 700, and FIG. 20 is a cross-sectional view of the connector 700 from the perspective of line 20-20 in FIG. 18. FIG. 21 is a top view of the connector 700 coupled to the fluid reservoir 600 in an unlocked state, FIG. 22 is a top view of the connector 700 coupled to the fluid reservoir 600 in a locked state, FIG. 23 is a top perspective view of the connector 700 coupled to the fluid reservoir 600 in the locked state, and FIG. 24 is a cross-sectional view of the connector 700 and a portion of the fluid reservoir 600, from the perspective of line 24-24 in FIG. 22.

The infusion set connector 700 is one element of an infusion set component (as described above with reference to FIG. 1). Accordingly, the infusion set connector 700 can include or cooperate with a conduit 702, such as a length of flexible tubing. The proximal end of the conduit 702 is physically and fluidly coupled to the infusion set connector 700, and the distal end of the conduit 702 is physically and fluidly coupled to an infusion unit, which may similar to the infusion unit 112 shown in FIG. 1. As described above with reference to FIG. 1, the conduit 702 can include or cooperate with a cannula that is designed and configured for insertion at the desired infusion site of the user. In this regard, the infusion unit and the cannula can be configured in accordance with established principles and technologies, and the manner in which the cannula is inserted into the user's skin can follow established and conventional methodologies. The infusion unit and cannula are not shown in FIGS. 17-24 for the sake of clarity and for ease of description.

As its name implies, the needleless infusion set connector 700 does not rely on a needle to establish a fluid connection with the fluid reservoir 600. Instead, the connector 700 cooperates with the fitting section 604 of the fluid reservoir 600 to manipulate and move the actuator 624, which in turn establishes a fluid flow path from the fluid chamber 636, through the fitting section 604, through the connector 700, and into the conduit 702. The illustrated embodiment of the connector 700 generally includes, without limitation: a main body section 706, an outlet port 708 defined in the main body section 706, and an engagement structure 710 that is suitably configured to receive the actuator 624 of the fluid reservoir 600 when the connector 700 is coupled to the reservoir 600.

The main body section 706 can be fabricated from molded plastic, as a single-piece component or a multi-part assembly. The main body section 706 has a generally annular shape, with an interior 714 that is shaped, sized, and configured to receive the fitting section 604 of the fluid reservoir 600. The interior 714 is generally defined by an inner wall 716 of the main body section 706. The outer wall 718 of the main body section 706 surrounds the inner wall 716. In certain embodiments, the connector 700 includes a housing lock structure that is suitably configured to removably couple the connector 700 to the housing 642 of the fluid infusion device, and/or a reservoir lock structure that is suitably configured to removably couple the connector 700 to the fluid reservoir 600. The housing lock structure can be designed to inhibit axial movement of the connector 700 relative to the housing, to inhibit rotation of the connector 700 relative to the fluid reservoir 600, or both. Similarly, the reservoir lock structure can be designed to inhibit axial movement of the connector 700 relative to the fluid reservoir 600, to inhibit rotation of the connector 700 relative to the fluid reservoir 600, or both.

In certain embodiments, the outer wall 718 can include or cooperate with two locking prongs 720 that extend away from the primary section of the outer wall 718, wherein the locking prongs 720 represent one possible implementation of the housing lock structure. The locking prongs 720 engage corresponding structure of the housing 642 and/or the collar 646 to lock the angular position of the connector 700 relative to the fluid reservoir 600. Referring to FIGS. 13, 14, and 21-23, the illustrated embodiment of the collar 646 cooperates with the locking prongs 720 to maintain the connector 700 in the proper position. In this regard, the collar 646 can include tabs 724 (or detents) that are shaped, sized, and located to engage with the locking prongs 720. The angled surfaces of the locking prongs 720 allow the prongs 720 to slide past the tabs 724 and snap back into place, as depicted in FIG. 22 and FIG. 23. The user can squeeze the sides of the locking prongs 720 inward to disengage them from the tabs 724, which allows the connector 700 to be rotated for removal.

In certain embodiments, the reservoir lock structure of the connector 700 is implemented as a plurality of tabs 728 located on the inner wall 716. Referring to FIG. 10 and FIG. 16, the tabs 728 are shaped, sized, and positioned for compatibility with the coupling structure 618 that resides on the fitting section 604 of the fluid reservoir 600. In this regard, the connector 700 is manipulated such that the tabs 728 can be fitted into the slots formed in the coupling structure 618. Thereafter, rotation of the connector 700 causes the tabs 728 to move under the rim of the coupling structure 618, which in turn retains the connector 700 in the desired axial position relative to the fluid reservoir 600.

The outlet port 708 serves as a fluid pathway that leads into the conduit 702 (refer to FIGS. 19, 20, and 24). The outlet port 708 has an opening 732 in the inner wall 716, as best shown in FIG. 19. The opening 732 leads to a structural passageway that runs between the inner wall 716 and the outer wall 718. The conduit 702 can be coupled to the outlet port 708 to create a continuous flow path that exits the connector 700. Thus, the conduit 702 remains in fluid communication with the outlet port 708, and the conduit 702 extends from the main body section 706 to the desired infusion site.

The engagement structure 710 of the connector 700 is shaped, sized, and positioned to receive the actuator 624 when the connector 700 is coupled to the fluid reservoir 600 (see FIGS. 21-24). For this particular embodiment, the engagement structure 710 is realized as a slot or keyway formed in the inner wall 716 of the connector 700. As shown in FIG. 18, the keyway spans the entire height of the inner wall 716 to accommodate insertion of the actuator 624 into the keyway as the connector 700 is installed onto the fitting section 604 of the fluid reservoir 600. In this regard, the exemplary embodiment of the actuator 624 can be realized as a key that mates with the engagement structure 710 when the connector 700 is coupled to the fluid reservoir 600. Notably, the engagement structure 710 (the keyway) rotates in concert with rotation of the connector 700. In alternative embodiments, the actuator 624 can include a keyway and the engagement structure 710 can include a key that mates with the keyway. In this alternate embodiment, the key of the connector 700 is received by the keyway of the fluid reservoir 600, and the key of the connector 700 rotates in concert with the connector 700.

FIG. 16 shows the fluid infusion device 640 after the fluid reservoir 600 has been installed and locked in place, and with the plug component 606 in the closed position. FIG. 21 shows the fluid infusion device 640 after the connector 700 has been coupled to the fluid reservoir 600 (and before rotation of the connector 700). FIG. 22 shows the fluid infusion device 640 after rotation of the connector 700 from its initial position to its final and locked position. Notably, rotation of the connector 700 relative to the fluid reservoir 600 from the unlocked position (FIG. 21) to the locked position (FIG. 22) causes the plug component 606 to rotate within the cavity defined by the sidewall structure 614. More specifically, rotation of the connector 700 moves the actuator 624, which in turn rotates the plug component 606 from the closed position (FIG. 21) to the open position (FIG. 22).

Rotating the connector 700 to the locked position establishes the fluid flow path from the fluid chamber 636 to the conduit 702 (see FIG. 24). More specifically, when the connector 700 is in the locked position, the outlet port 708 of the connector 700 is fluidly coupled to the through-hole 616 formed in the fitting section 604 of the fluid reservoir 600. This arrangement results in fluid communication between the conduit 702 and the fluid chamber 636, via the fluid flow path 626 defined in the plug component 606. As shown in FIG. 24, the outlet opening 632 is brought into alignment with the through-hole 616 when the plug component 606 is in the open/locked position, which allows fluid to flow out of the fluid reservoir 600. The fluid that exits the reservoir 600 directly or indirectly flows into the opening 732 of the connector 700.

In accordance with the illustrated embodiment, the assembly (which is formed by coupling the connector 700 to the fluid reservoir 600) includes a transition chamber 740 that resides between the sidewall structure 614 of the reservoir 600 and the inner wall 716 of the connector 700. The transition chamber 740 can be a thin annular space between the reservoir 600 and the connector 700, or it can be shaped and sized in any desired manner. Although not shown, the fluid reservoir 600 and/or the connector 700 can include appropriate sealing features, elements, or components that inhibit leakage of fluid from the transition chamber 740. In accordance with alternative embodiments, the outlet opening 632 of the reservoir 600 is in direct fluid communication with the outlet port 708 of the connector 700, which obviates the need for the transition chamber 740.

The needleless infusion set connector 700 has a number of practical benefits. For example, the absence of a needle makes the connector 700 safer and eliminates risk that might otherwise be associated with the use of a sharp needle. Moreover, the manufacturing cost of the connector 700 is less than a counterpart connector that includes a needle. Furthermore, the connector 700 is compatible with the automated pump-fill methodology described above and, therefore, it facilitates quicker and easier filling of the fluid reservoir 600.

Infusion Set Connector with Needle

As an alternative to the needleless infusion set connector 700, an exemplary embodiment of the fluid infusion system can utilize a needled infusion set connector that is compatible with a fluid reservoir of the type described above. In this regard, the needled infusion set connector can be physically and fluidly coupled to the fluid reservoir after the reservoir has been properly installed and locked into the housing of the fluid infusion device. In contrast to many conventional infusion set connectors, the needled infusion set connector described here mates only with the fluid reservoir, and it does not physically attach to the housing of the fluid infusion device.

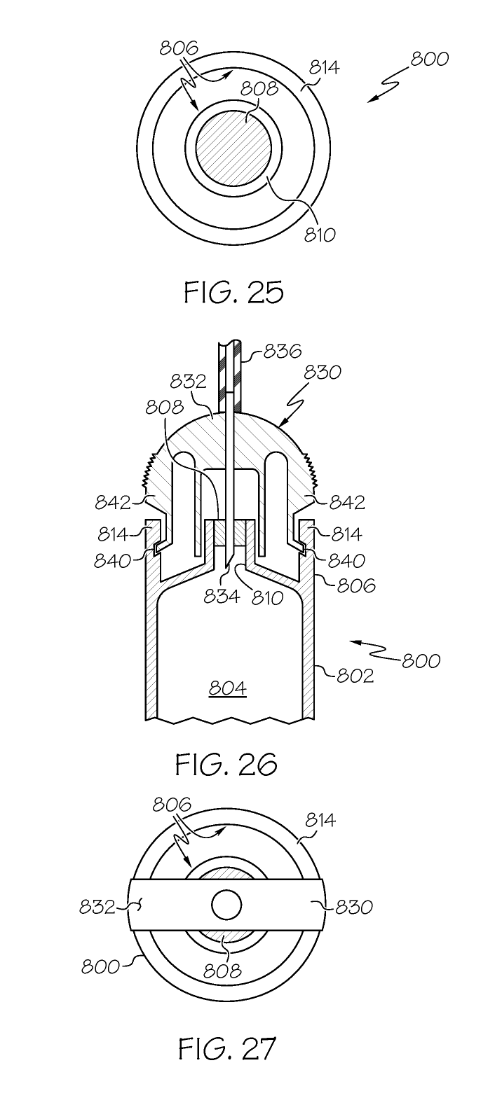

FIG. 25 is a top view of an exemplary embodiment of a fluid reservoir 800 suitable for use with a fluid infusion device. FIG. 26 is a partial cross-sectional view of the fluid reservoir 800 with a needled infusion set connector, and FIG. 27 is a top view of the assembly shown in FIG. 26. The fluid reservoir 800 shares some of the features, structures, and functionality of the fluid reservoirs described in more detail above, and common aspects will not be redundantly described in this section. The fluid reservoir 800 generally includes, without limitation: a primary body section 802 having a fluid chamber 804 defined therein; a fitting section 806 extending from the primary body section 802; and a septum 808. Although not shown in FIGS. 25-27, the fluid reservoir 800 includes a piston inside the primary body section 802, wherein the piston can be actuated by the fluid infusion device as needed. Moreover, the fluid reservoir 800 preferably includes a suitably configured housing lock structure (as previously described) that removably couples the fluid reservoir 800 to the housing of the fluid infusion device. As explained above, the housing lock structure secures the fluid reservoir 800 in place to inhibit axial movement of the fluid reservoir 800 relative to the housing. This locking arrangement allows the user to install the needled infusion set connector quickly and safely. The housing lock structure of the fluid reservoir 800 can be realized in accordance with any of the embodiments described in the preceding sections of this disclosure.

The fluid chamber 804 terminates at a neck section 810 of the fluid reservoir 800. The neck section 810 (or a portion thereof) can also form a part of the fitting section 806, as shown in FIG. 26. The neck section 810 extends from the primary body section 802, and it can be centered about the major longitudinal axis of the fluid reservoir 800 (see FIG. 25). The septum 808 is positioned at or near the top of the neck section 810, and the septum 808 is shaped, sized, and configured to seal the neck section 810. The septum 808 is formed from a self-healing material, such as silicone, that accommodates piercing by a hollow needle for purposes of filling the fluid chamber 804 and for purposes of delivering medication fluid from the fluid chamber 804.

The fitting section 806 extends from the primary body section 802. In certain embodiments, the fitting section 806 protrudes upward such that at least a portion of it remains accessible when the fluid reservoir 800 is installed in the housing of the fluid infusion device. The fitting section 806 includes an outer wall 814 surrounding the neck section 810. The illustrated embodiment employs a continuous outer wall 814 that completely encircles the neck section 810. In alternative embodiments, however, the outer wall 814 can be discontinuous. In yet other embodiments, a plurality of upstanding tabs or equivalent locking structures can be used instead of the outer wall 814.

The fitting section 806 includes or cooperates with a suitably configured connector lock structure that mates with compatible structure of the needled infusion set connector. The embodiment presented here employs a connector lock structure that is integrated with the fitting section 806. The connector lock structure mates and cooperates with a reservoir lock structure of the infusion set connector to form a threaded engagement, a snap-lock engagement, a bayonet mount engagement, or the like. The locking interface is described in more detail below.

The fluid reservoir 800 is compatible with the needled infusion set connector 830 shown in FIGS. 26 and 27. The illustrated embodiment of the connector 830 generally includes, without limitation: a main body section 832; a hollow needle 834 supported by the main body section 832; a conduit 836; and a reservoir lock structure. The proximal end of the conduit 836 is attached to the main body section 832. Thus, the conduit 836 extends from the main body section 832, and it fluidly communicates with the hollow needle 834. Although not shown in FIG. 26 or FIG. 27, the distal end of the conduit 836 is physically and fluidly connected to a suitably configured infusion unit (of the type described above with reference to FIG. 1).

The reservoir lock structure can be integrated with or coupled to the main body section 832. The reservoir lock structure is designed to cooperate with the connector lock structure of the fluid reservoir 800, such that the connector 830 can be temporarily locked to the fluid reservoir 800. As mentioned briefly above, the reservoir lock structure mates and cooperates with the connector lock structure of the fluid reservoir 800 to form a threaded engagement, a snap-lock engagement, a bayonet mount engagement, or the like. Referring to FIG. 26, the illustrated embodiment of the assembly employs a simple snap-lock engagement. In this regard, the connector lock structure of the fluid reservoir 800 can be implemented as at least one detent 840, one or more grooves, a keyway, or similar structure integrated with or formed in the outer wall 814 of the fitting section 806, and the reservoir lock structure of the connector 830 can be implemented as a plurality of locking arms 842, tabs, flanges, protrusions, or similar snap-fitting structure integrated with the main body section 832 of the connector 830. The locking arms 842 flex inward and automatically snap into place to engage the detents 840 when the connector 830 is installed and forced onto the fitting section 806. The user can squeeze the locking arms 842 inward to release them from the detents 840, which allows the connector 830 to be removed from the fluid reservoir 800. In alternative embodiments, the connector 830 can include at least one detent formed in the main body section 832, wherein the at least one detent receives locking arms or other snap-fitting structure formed in the outer wall 814 of the fluid reservoir 800. Notably, the connector 830 can be removably locked to the fluid reservoir 800 in a way that does not require any physical coupling to the housing of the fluid infusion device.

The shape, size, and arrangement of the main body section 832 (of the connector 830) and the fitting section 806 (of the fluid reservoir 800) are configured such that the end of the hollow needle 834 is automatically aligned with the septum 808 when the connector 830 engages the fitting section 806. The hollow needle 834 pierces the septum 808 when the connector 830 is urged into, and coupled to, the fitting section 806. FIG. 26 depicts the connector 830 in the locked position. When in the locked state, the hollow needle 834 protrudes through the septum 808 and is held in fluid communication with the fluid chamber 804. The hollow needle 834 is extracted from the septum 808 when the connector 830 is unlocked and removed, and the septum 808 naturally heals itself to seal the neck section 810 of the fluid reservoir 800.

The fitting section 806 and the connector lock structure of the fluid reservoir 800 can also be designed and manufactured to be compatible with a reservoir filling component (e.g., a transfer guard as described above with reference to FIG. 3) to facilitate filling of the fluid chamber 804 with the desired medication fluid. As explained above, certain preferred embodiments support an automatic pump-fill procedure, where the fluid reservoir 800 is installed and locked into the housing to allow the infusion device to engage the piston as needed to fill the fluid chamber 804. After filling the reservoir 800, the filling component can be removed and replaced with the needled infusion set connector 830, and the fluid infusion device can be operated to prime the fluid flow path and to otherwise prepare the system for normal operation.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A fluid reservoir for a fluid infusion device that delivers medication fluid to a user, the fluid reservoir comprising:
a primary body section having a fluid chamber defined therein;
a fitting section extending from the primary body section;
a housing lock structure to removably couple the fluid reservoir to a housing of the fluid infusion device, the housing lock structure inhibiting axial movement of the fluid reservoir relative to the housing; and
a piston located within the fluid chamber, the piston being axially movable relative to the primary body section, the piston comprising a tip section facing the fluid chamber and a base section opposite the tip section, the base section comprising an actuator lock structure to removably couple the piston to a piston actuator of the fluid infusion device.

2. The fluid reservoir of claim 1, wherein the housing lock structure comprises a first threaded structure that mates with a threaded feature of the housing.

3. The fluid reservoir of claim 2, wherein the actuator lock structure comprises a second threaded structure that mates with a threaded distal end of the piston actuator.

4. The fluid reservoir of claim 3, wherein:
the second threaded structure is formed as an internally threaded cavity of the piston; and
the threaded distal end of the piston actuator is formed as an externally threaded tip.

5. The fluid reservoir of claim 3, wherein thread pitch of the first threaded structure is different than thread pitch of the second threaded structure to facilitate coupling of the piston actuator to the piston in response to coupling the fluid reservoir to the housing.

6. The fluid reservoir of claim 1, wherein the actuator lock structure comprises a snap-fitting receptacle that mates with a compatible snap-fitting neck structure on a distal end of the piston actuator.

7. The fluid reservoir of claim 1, wherein the actuator lock structure comprises a snap-fitting neck structure that mates with a compatible snap-fitting receptacle on a distal end of the piston actuator.

8. The fluid reservoir of claim 1, further comprising a retaining structure formed at a base end of the primary body section, the retaining structure inhibiting removal of the piston from the fluid chamber.

9. A fluid infusion device to deliver medication fluid to a user, the fluid infusion device comprising:
a housing having a reservoir cavity defined therein to receive a fluid reservoir containing the medication fluid;
a reservoir lock structure integrated with or coupled to the housing, wherein the reservoir lock structure mates with a compatible housing lock structure of the fluid reservoir to inhibit axial movement of the fluid reservoir relative to the housing; and
a piston actuator comprising a distal end that includes a piston lock structure to removably couple the piston actuator to a piston of the fluid reservoir.

10. The fluid infusion device of claim 9, wherein the reservoir lock structure cooperates with the housing lock structure of the fluid reservoir to form a threaded engagement.

11. The fluid infusion device of claim 10, wherein the piston lock structure comprises locking threads formed in the distal end of the piston actuator.

12. The fluid infusion device of claim 11, wherein the distal end of the piston actuator is formed as an externally threaded tip that cooperates with an internally threaded cavity of the piston to form a threaded engagement.

13. The fluid infusion device of claim 9, wherein the piston lock structure comprises a snap-fitting receptacle that mates with a compatible snap-fitting neck structure on a base section of the piston.

14. The fluid infusion device of claim 9, wherein the piston lock structure comprises a snap-fitting neck structure that mates with a compatible snap-fitting receptacle on a base section of the piston.

15. An assembly for a fluid infusion device that delivers medication fluid to a user, the assembly comprising:
a fluid reservoir comprising:
a primary body section having a fluid chamber defined therein;
a fitting section extending from the primary body section;
a housing lock structure to removably couple the fluid reservoir to a housing of the fluid infusion device, the housing lock structure inhibiting axial movement of the fluid reservoir relative to the housing; and
a piston located within the fluid chamber, the piston being axially movable relative to the primary body section, the piston comprising a tip section facing the fluid chamber and a base section opposite the tip section, the base section comprising an actuator lock structure; and
a piston actuator to control axial movement of the piston within the fluid chamber, the piston actuator comprising a piston lock structure that cooperates with the actuator lock structure of the piston to removably couple the piston to the piston actuator.

16. The assembly of claim 15, wherein:
the actuator lock structure comprises threads integrally formed in the base section of the piston; and
the piston lock structure comprises threads integrally formed in a distal end of the piston actuator, the threads formed in the base section mating with the threads formed in the distal end to form a threaded engagement.

17. The assembly of claim 16, wherein:
the actuator lock structure is formed as an internally threaded cavity of the piston; and
the piston lock structure is formed as an externally threaded tip of the piston actuator.

18. The assembly of claim 15, wherein:
the actuator lock structure comprises a snap-fitting receptacle; and
the piston lock structure comprises a snap-fitting neck structure formed on a distal end of the piston actuator, the snap-fitting neck structure mating with the snap-fitting receptacle to form a releasable snap-fit engagement.

19. The assembly of claim 15, wherein the fluid reservoir further comprises a retaining structure formed at a base end of the primary body section, the retaining structure inhibiting removal of the piston from the fluid chamber.
